(19) United States
(12) Patent Application Publication
(10) Pub. No.: US 2024/0321394 A1
(43) Pub. Date: Sep. 26, 2024

(54) Whole-Slide Image Classification and Gene Profile Prediction Using Machine Learning

(71) Applicant: Mayo Foundation for Medical Education and Research, Rochester, MN (US)

(72) Inventors: Hamid R. Tizhoosh, Rochester, MN (US); Amir S. Kordbacheh, Waterloo (CA)

(21) Appl. No.: 18/615,855

(22) Filed: Mar. 25, 2024

Related U.S. Application Data
(60) Provisional application No. 63/492,212, filed on Mar. 24, 2023.

(51) Int. Cl. G16B 25/10; G06T 7/00

(52) U.S. Cl. CPC G16B 25/10; G06T 7/0012; G06T 2207/20081; G06T 2207/30072

(57) ABSTRACT

A complete gene expression profile is predicted from digital pathology images, such as a whole-slide images, using an attention-based machine learning model that is based on a transformer encoder architecture. While predicting gene profiles, the machine learning model simultaneously learns a whole-slide image representation, and thus also outputs classified feature data that indicate a classification of the whole-slide images.

### BACKGROUND

Deep learning methods are widely applied in digital pathology to address clinical challenges such as prognosis and diagnosis. In recent applications, deep models have also been used to extract molecular features from whole slide images. Although molecular tests carry rich information, they are often expensive, time-consuming, and require additional tissue to sample.

### SUMMARY OF THE DISCLOSURE

In one aspect, the present disclosure provides a method for predicting gene profile data from a whole-slide image using a computer system. The method includes accessing whole-slide image (WSI) data with the computer system, where the WSI data include whole-slide images of a histopathology sample. A machine learning model is also accessed with the computer system, where the machine learning model has been trained on training data to predict gene profile data and to classify whole-slide images. The WSI data are input to the machine learning model using the computer system, generating as outputs gene profile data and classified WSI data. The gene profile data are indicative of a predicted gene profile for the histopathology sample and the classified WSI data are indicative of a classification of the whole-slide images of the histopathology sample as one of different disease classifications. The gene profile data and classified WSI data may be presented to a user by the computer system.

It is another aspect of the present disclosure to provide a method for generating complete genome profile data for a histopathology sample. The method includes accessing a whole-slide image with a computer system, where the whole-slide image depicts the histopathology sample. An attention-based transformer encoder model is also accessed with the computer system, and the whole-slide image is input to the attention-based transformer encoder model, generating gene prediction data as an output. The gene prediction data include a complete genome profile for the histopathology sample. The gene prediction data may be presented to a user.

It is yet another aspect of the present disclosure to provide a method for training a transformer encoder model to generate predicted gene profile and classified feature data from a whole-slide image. The method includes accessing whole-slide image data with a computer system, where the whole-slide image data include whole-slide images that depict histopathology samples. The method also includes accessing gene expression data with the computer system, where the gene expression data include gene expressions corresponding to the histopathology samples depicted in the whole-slide images. The whole-slide image data and the gene expression data are assembled into at least a training dataset using the computer system. A transformer encoder model is then accessed with the computer system and trained on the training dataset.

### BRIEF DESCRIPTION OF THE DRAWINGS

FIG. 1 is an example of a tRNAsformer model architecture, which implements an attention-based transformer encoder model architecture that output

FIG. 2 is an example structure for a multilayer perceptron block that may form a part of the tRNAsformer model architecture.

FIG. 3 illustrates an example workflow for preprocessing whole-slide image data and inputting the whole-slide image data to a tRNAsformer model to generate predicted gene profile data and classified feature data as classified whole-slide image data.

FIG. 4 is a flowchart setting forth the steps of an example method for generating gene profile data in addition to classified whole-slide image data by inputting whole-slide image data to a machine learning model implementing an attention mechanism-based transformer encoder model.

FIG. 5 is a flowchart setting forth the steps of an example method for training a machine learning model (e.g., the tRNAsformer model of FIG. 1) on training data, such that the machine learning model is trained to receive embedded instances of whole-slide images as input data in order to generate predicted gene profile data and classified whole-slide image data as outputs.

FIG. 6 illustrates a process for preprocessing a whole-slide image patch to generate a bag of instances as clustered whole-slide image data.

FIG. 7 is a block diagram of an example system for predicting gene profile data and classifying whole-slide image data.

FIG. 8 is a block diagram of example components that can implement the system of FIG. 7.

### DETAILED DESCRIPTION

Described here are systems and methods for predicting a complete gene expression profile from digital pathology images, such as a whole-slide image (“WSI”), using an attention-based machine learning model that is based on a transformer architecture. Advantageously, while predicting gene profiles, the machine learning model is also capable of learning WSI representation.

As will be described, the proposed machine learning model framework can learn reliable internal representations for massive archives of pathology slides that match or outperform the performance of existing classification and search algorithms. The disclosed machine learning model framework can provide for improved gene expressions prediction from WSIs (e.g., hematoxylin and eosin (“H&E”) slide images). For instance, by employing a balanced architecture, the disclosed models may outperform existing topologies in both tasks simultaneously. It is another advantage of the disclosed machine learning models that, instead of requiring a large number of samples, the gene expression profile can be predicted based on only a small number of samples. Having a fewer number of parameters makes the machine learning model more efficient during training and testing.

In some implementations, the machine learning models described in the present disclosure may be referred to as a tRNAsformer model. As an example, the tRNAsformer model may include an attention-based topology that can learn both to predict gene profile data (e.g., the bulk RNA-seq) from an image and represent the WSI of a glass slide simultaneously. The tRNAsformer model may use multiple instance learning (“MIL”) to solve a weakly supervised problem the pixel-level annotation is not available for an image. The disclosed tRNAsformer model can assist as a computational pathology tool to facilitate a new generation combining of tissue morphology and the molecular fingerprint of the biopsy samples.

By incorporating the attention mechanism and the transformer design, the tRNAsformer model can provide more precise predictions for gene expressions from a WSI. Additionally, the tRNAsformer model is capable of gene profile (e.g., bulk RNA-seq) prediction while having fewer hyperparameters. As another advantage, the tRNAsformer model can learn compact representation for a WSI using the molecular signature of the tissue sample. As a result, the proposed techniques may learn a diagnostically relevant representation from an image by integrating gene information in a multimodal approach.

As another advantage, the transformer design allows for more efficient and precise processing of a collection of samples. Accordingly, the need for costly and time-consuming pixel-by-pixel human annotations can be significantly reduced when using the tRNAsformer models described in the present disclosure.

Moreover, sampling and embedding image tiles using pre-trained convolutional neural network (“CNN”) models offers several advantages. As one example, by training on large image datasets, deep CNNs can be exploited to create rich intermediate embeddings from image samples. As another example, working with embedded sampled instances is less computationally expensive in comparison with treating each WSI as an instance. In an example configuration, a tRNAsformer model can have upwards of 60% fewer hyperparameters in comparison with MLP-based models. Additionally, the tRNAsformer model can be about 72% and 15% faster than MLP-based model during training and validation, respectively. By augmenting data, bootstrapping can meet the requirement for big datasets for training deep models, and by diversifying the instances in a bag, bootstrapping at test time can reduce noise.

In contrast to methods where a spatial transcriptomics dataset was available, the systems and methods described in the present disclosure use bulk RNA-seq data. As a result, the models described in present disclosure employ a weaker type of supervision, as they learn internal representation using a combination of a primary diagnosis and a bulk RNA-seq associated with a WSI. This is more in line with current clinical practice, which generally collects bulk RNA sequences rather than spatial transcriptomic data. Furthermore, the tRNAsformer model can handle the problem by treating a WSI in its entirety, whereas previous methods separate each tile and estimate the gene expression value for it, which can result in ignoring the dependencies between tiles. In addition, the tRNAsformer model can learn WSI representation by learning a pixel-to-gene translation.

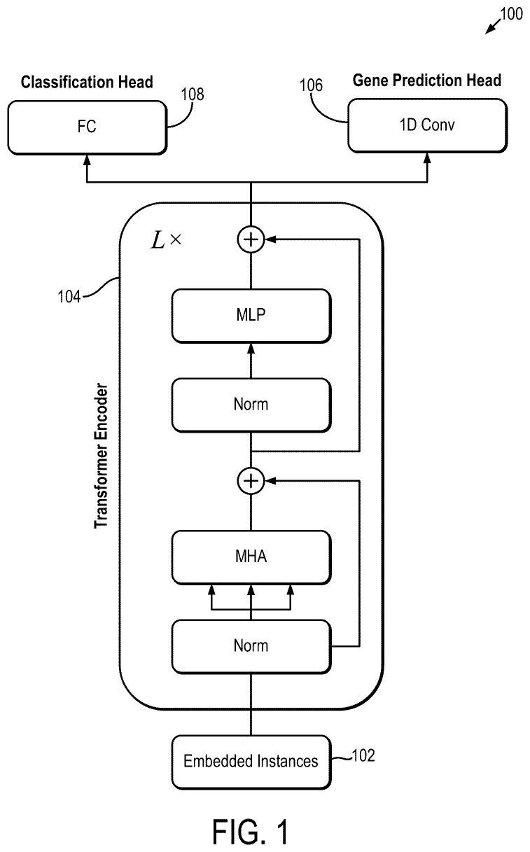

An example model architecture for a tRNAsformer model is illustrated in FIG. 1. In the illustrated example, a multi-head attention-based transformer encoder model is implemented. It will be appreciated that other transformer models may also be implemented. The illustrated model 100 receives input data at an input layer 102 as embedded instances of WSI data (e.g., bags of instances, or tiles, extracted from WSI data). The input data are input to L transformer encoder layers 104 to generate a first output 106 as gene prediction data output from a gene prediction head and a second output 108 as classification data output from a classification head.

In the illustrated example, the transformer encoder learns an embedding (also known as the class token) for the input by treating it as a sequence of feature instances associated with each WSI. The transformer encoder learns internal embeddings for each instance while learning the class token that represents the bag, or WSI. The transformer encoder blocks include layernorm, multi-head attention (MHA), multi-layer perceptron (MLP) block, and residual skip connections. Because it is a multi-head self-attention module, the output embedding of the first layernorm is provided to the multi-head attention as the query, key, and value. Each model can have L blocks of transformer encoder. As a non-limiting example, the model may include L=1 transformer encoder blocks, L=8 transformer encoder blocks, L=12 transformer encoder blocks, or the like. More generally, the model may include between 1-12 transformer encoder blocks as an example. The classification head transforms the internal representation to the number of classes, whereas the gene prediction head maps it to the number of genes.

The classification head, which is a linear layer in the illustrated example, receives the WSI representation, c. The WSI representation is projected using a linear layer to the WSI's score, ŷ. The tRNAsformer model then uses cross-entropy loss, for example, between the predicted score ŷ and the WSI's true label y to learn the primary diagnosis. The use of the transformer encoder and the classification head enables the learning of the WSI's representation while training the model.

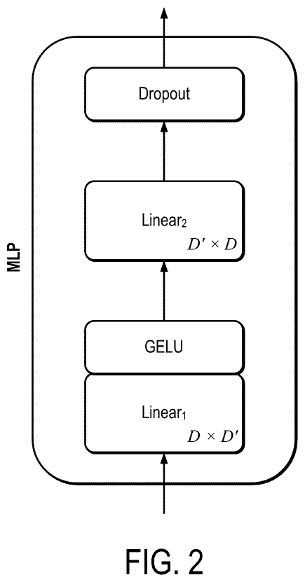

Considering a bag X=[x₁, x₂, . . . , xₖ], where xᵢ∈ [symbol] ^{d}, i=1, . . . , k are embedded tiles (e.g., tiles embedded by a CNN such as a DenseNet-121 CNN), an L-layer standard Transformer can be defined as:

$${z}_{0} = [ {x}_{class} ; {x}_{1} E ; {x}_{2} E ; … ; {x}_{k} E ] + {E}_{pos} , E ∈ {ℝ}^{d \times D} , {E}_{pos} ∈ {ℝ}^{( k + 1 ) \times D}$$

$${z}_{ℓ}^{′} = MSA ( LN ( {z}_{ℓ - 1} ) ) + {z}_{ℓ - 1} , ℓ = 1 , … , L$$

$${z}_{ℓ} = MLP ( LN ( {z}_{ℓ}^{′} ) ) + {z}_{ℓ}^{′} , ℓ = 1 , … , L$$

$$c = LN ( {z}_{L}^{0} ) ,$$

$$\hat{y} = L ( c ) ,$$

where MSA, LN, MLP, L, E, and Eₚₒₛ are multi-head self-attention (“MSA”), layernorm (“LN”), multi-layer perceptron block (“MLP”), linear layer (“L”), tile embedding projection (“E”), and position embedding (“Eₚₒₛ”). The variables E and Eₚₒₛ are learnable. The layernorm applies normalization over a minibatch of inputs. In layernorm, the statistics may be calculated independently across feature dimensions for each instance (e.g., tile) in a sequence (e.g., a bag of tiles). The multi-layer perceptron block may be made of two linear layers followed by a dropout layer. In such instances, the first linear layer may include a Gaussian error linear unit (“GELU”) activation function, or another suitable activation function. The embedding may be projected to a higher dimension in the first layer and then mapped to its original size in the second layer. FIG. 2 shows an example structure of an MLP block in a transformer encoder. In this example, the letter D refers to the size of internal representation in the transformer encoder, and D′/D is the MLP ratio

The remaining internal embeddings may be passed to a dropout layer followed by a 1D convolution layer for the gene prediction head. As an example, the gene prediction head uses a dropout layer and 1D convolution layer as the output layer. The first two layers include a transformer encoder to capture the relationship between all instances. As the model produces one prediction per gene per instance, an aggregation strategy may be adapted for computing the gene prediction for each WSI. As a non-limiting example, a random number, n, may be sampled at each iteration and each gene's prediction may be calculated by averaging the top-n predictions by tiles in a WSI (bag). This approach acts as a regularization technique and decreases the chance of overfitting. In a non-limiting example, for bags having 49 tile embeddings in each bag, n may be randomly selected from {1, 2, 5, 10, 20, 49}. For a randomly selected n during training, the gene prediction outcome can be written as,

$$s = Conv 1 D ( {z}_{L}^{1 : end} ) ,$$

$$S ( n ) = {\sum }_{i = 1}^{n} \frac{{s}^{i}}{n} ,$$

where z_{L}^{1:end} ∈ [symbol] ^{D×k}, s∈ [symbol] ^{D×k}, and S(n)∈ [symbol] _{d}_{_{g}} are the internal embeddings excluding the class token, the tile-wise gene prediction, and slide-level gene expression, respectively. During the test the final prediction, S, may be calculated as an average of all possible values for n as,

$$S = {\sum }_{i = 1}^{k} \frac{S ( i )}{i} .$$

The mean squared error loss function can be employed to learn gene predictions. As an example, the total loss for the tRNAsformer model may be computed as,

$$\begin{matrix}{ℒ}_{total} ( θ ) = {ℒ}_{classification} ( θ ) + γ {ℒ}_{prediction} ( θ ) + λ {ℒ}_{regularization} ( θ ) , \\ = \frac{1}{B} {\sum }_{i = 1}^{B} ( - {y}_{i} log ( {\hat{y}}_{i} ) + γ \vert {y}_{i}^{g} - {S}_{i} \vert ) + λ {\vert θ \vert}_{2}^{2} ,\end{matrix}$$

where θ, λ, γ, B, y^{g} are the model parameters, weight regularization coefficient, hyperparameter for scaling the losses, number of samples in a batch, and true bulk RNA-seq associated with the slides, respectively.

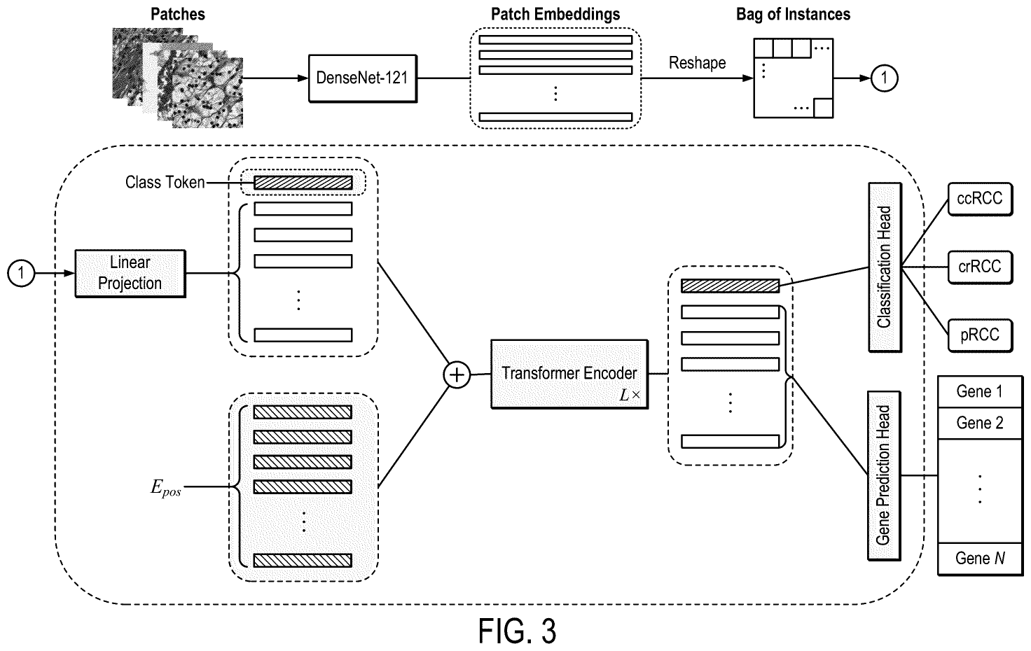

A summary of implementing the proposed approach is illustrated in FIG. 3. Tiles are selected from spatial clusters in a WSI and are embedded with a CNN, such as a DenseNet-121 CNN. As an example, 49 tiles of size 224×224×3 selected from 49 spatial clusters in a WSI are embedded with a DenseNet-121. The outcome is a matrix of size 49×1024 as DenseNet-121 has 1024 deep features after the last pooling. Then the matrix is reshaped and rearranged to 224×224 matrix in which each 32×32 block corresponds to a tile embedding 1×1024. A 2D convolution is applied to the embedded instances, such as a 2D convolution with kernel 32, stride 32, and 384 kernels, each 32×32 block has linearly mapped a vector of 384 dimensional. Next, a class token is concatenated with the rest of the tile embeddings, and Eₚₒₛ is added to the matrix before entering the L encoder layers. The first row of the outcome, which is associated with the class token, is fed to the classification head. The rest of the internal embeddings that are associated with all tile embeddings are passed to the gene prediction head. The parts of the model with learnable variables that can be trained during training are shown in purple.

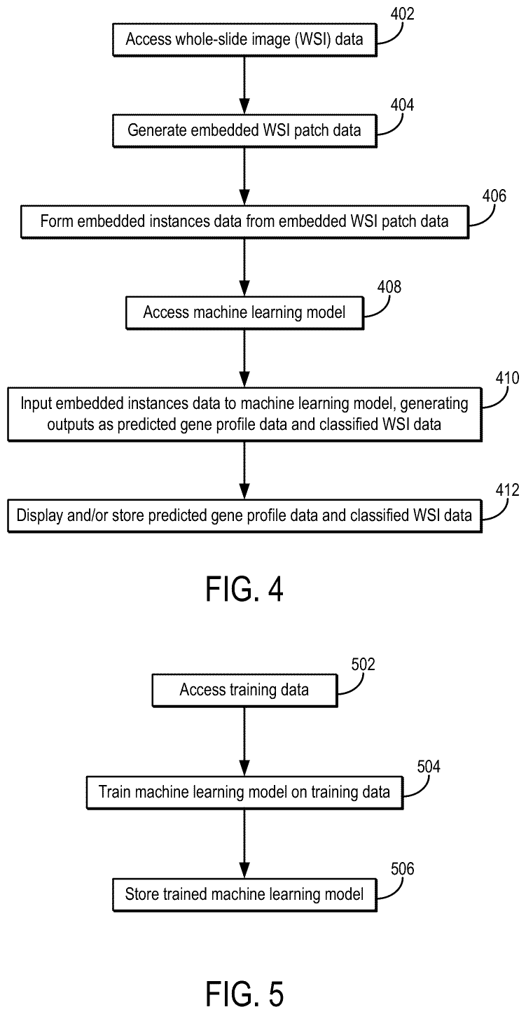

Referring now to FIG. 4, a flowchart is illustrated as setting forth the steps of an example method for generating gene profile data in addition to classified WSI data by inputting WSI data to a machine learning model implementing an attention mechanism-based transformer encoder model. As described above, the machine learning model takes WSI data as input data and generates predicted gene profile data and classified WSI data as output data.

The method includes accessing whole-slide image (“WSI”) data with a computer system, as indicated at step 402. Accessing the WSI data may include retrieving such data from a memory or other suitable data storage device or medium. Additionally or alternatively, accessing the WSI data may include acquiring such data and transferring or otherwise communicating the data to the computer system. For example, WSI data can be acquired with a slide scanner or other suitable imaging system. In some instances, the WSI data may include whole-slide images that depict a histopathology sample (e.g., cell sample(s), tissue sample(s)). In other instances, the WSI data may include preprocessed whole-slide images, such as whole-slide images that have been divided into tiles or patches. The tiles may be collected as bags of instances, as described above. In some instances, the preprocessed whole-slide images may include embedded instances, as described above.

The WSI data are then processed to generate patch embeddings, as indicated at step 404. For example, as described above, the whole-slide images in the WSI data can be divided into tiles, or patches. These patches can then be input to a neural network, such as a DenseNet-121 CNN, to generate embedded WSI patch data. In these instances, the neural network can include a trained neural network that has been trained on training data to generate embeddings from whole-slide images, patches, or other WSI data. For example, the neural network may be trained on training data to generate patch embeddings from whole-slide image patches.

The patch embeddings are then formed into embedded instance data, as indicated at step 406. As an example, the embedded instance data can include a bag of instances, as described above in more detail. For instance, the embedded WSI patch data can be reshaped and/or rearranged into a matrix where each block corresponds to a tile/patch embedding.

A trained machine learning model (e.g., a neural network or other suitable machine learning model) is then accessed with the computer system, as indicated at step 408. Accessing the trained machine learning model may include accessing model parameters (e.g., weights, biases, or both) that have been optimized or otherwise estimated by training the machine learning model on training data. In some instances, retrieving the machine learning model can also include retrieving, constructing, or otherwise accessing the particular model structure or architecture to be implemented. For instance, data pertaining to the layers in a neural network architecture (e.g., number of layers, type of layers, ordering of layers, connections between layers, hyperparameters for layers) may be retrieved, selected, constructed, or otherwise accessed.

In general, the machine learning model is trained, or has been trained, on training data in order to predict gene profile data and classify WSI data, as described above.

The embedded instance data (e.g., the bag of instances formed in step 406) are then input to the trained machine learning model, generating output as predicted gene profile data and classified WSI data, as indicated at step 410.

As an example, the classified WSI data, which may also be referred to as classified feature data, may indicate the probability for a particular classification (i.e., the probability that the WSI data include patterns, features, or characteristics indicative of detecting, differentiating, and/or determining the severity of one or more medical conditions). The classified WSI data may indicate the probability of a particular classification for an entire whole-slide image, or alternatively may indicate the probability of a particular classification for one or more subregions of a whole-slide image. In this latter example, the classified WSI data may indicate the probability of a particular classification for each of a plurality of different subregions. In some instances, the classified WSI data may also indicate probabilities of particular classifications for one or more subregions in addition to a probability of a particular classification for the entire whole-slide image.

Additionally or alternatively, the classified WSI data may classify the WSI data as indicating a particular medical condition, such as by classifying the histopathology sample depicted in the whole-slide image(s) as belonging to a particular disease, subtype, or the like. In these instances, the classified WSI data can differentiate between different medical conditions. As a non-limiting example, for a histopathology sample from the kidney the classified WSI data may indicate a disease subtype, such as clear cell carcinoma, chromophobe type renal cell carcinoma, papillary carcinoma, or the like. The classified WSI data may classify an entire whole-slide image, may classify individual subregions within a whole-slide image, or both.

In still other examples, the classified WSI data may indicate a severity of a medical condition. For example, the classified WSI data may include a severity score that quantifies a severity of a medical condition.

The predicted gene profile data and classified WSI data generated by inputting the embedded instance data to the trained machine learning model(s) can then be displayed to a user, stored for later use or further processing, or both, as indicated at step 412.

Referring now to FIG. 5, a flowchart is illustrated as setting forth the steps of an example method for training one or more machine learning models (e.g., the tRNAsformer model described above) on training data, such that the one or more machine learning models are trained to receive embedded instances as input data in order to generate predicted gene profile data and classified WSI data as outputs. An example of the machine learning model architecture to be trained is described above with respect to FIGS. 1 and 2, and with respect to FIG. 3 illustrating components of the example model architecture that have learnable variables.

The method includes accessing training data with a computer system, as indicated at step 502. Accessing the training data may include retrieving such data from a memory or other suitable data storage device or medium. In general, the training data can include whole-slide images and gene profiles (e.g., RNA-seq data).

The method can include assembling training data from whole-slide images and gene profile data using a computer system. This step may include preprocessing the whole-slide images and/or gene profile data, and may also include assembling the whole-slide images and gene profile data into an appropriate data structure on which the machine learning algorithm can be trained.

As a non-limiting example, the training data may include WSI data such as H&E-stained formalin-fixed, paraffin-embedded (“FFPE”) diagnostic slides. For transcriptomic data, Fragments Per Kilobase of transcript per Million mapped reads upper quartile (“FPKM-UQ”) files can be utilized. In an example study, these data were split case-wise into training (80%), validation (10%), and test (10%) sets, respectively.

As mentioned above, the gene expression data may be preprocessed. In an example, FPKM-UQ files containing 60,483 Ensembl gene IDs. The genes with a median of zero across all kidney cases were excluded. As a result, the final gene expression vector was of size 31,793. The α→log₁₀ (1+a) transform was used to convert the gene expressions since the order of gene expression values changes considerably and can impact mean squared error only in the case of highly expressed genes.

As also mentioned above, the WSI data may also be preprocessed. In general, the size of digitized glass slides included in WSI data may be upwards of 100,000×100,000 in pixels, or even larger. As a result, processing an entire slide at once is challenging with standard computer hardware. These images are commonly divided into smaller, more manageable pieces known as tiles or patches. Furthermore, large WSI datasets are generally weakly labeled since pixel-level expert annotation is costly and labor-intensive. As a result, some of the tiles may not carry information that is relevant to the diagnostic label associated with the WSI. Consequently, multi-instance learning (“MIL”) may be suitable for this scenario. Instead of receiving a collection of individually labeled examples, the learner receives a set of labeled bags, each including several instances in MIL.

To make bags of instances, the first step is to figure out where the tissue boundaries are. For instance, tissue boundaries in each whole-slide image patch are identified. Whole-slide image patches are discarded when they have a percentage of pixels associated with tissue that is lower than a threshold value. The non-discarded whole-slide image patches (i.e., the whole-slide image patches having a percentage of pixels that is at or above the threshold) may then be input to a clustering algorithm to generate the bags of instances. As one example, the tissue region may be located at the thumbnail (1.25× magnification) while the background and the marker pixels are removed. Tiles of size 14-by-14 pixels were processed using the 1.25× tissue mask to discard those with less than 50% tissue. Note that 14-by-14 pixel tiles at 1.25× is equivalent to an area of 224×224 pixels at 20× magnification.

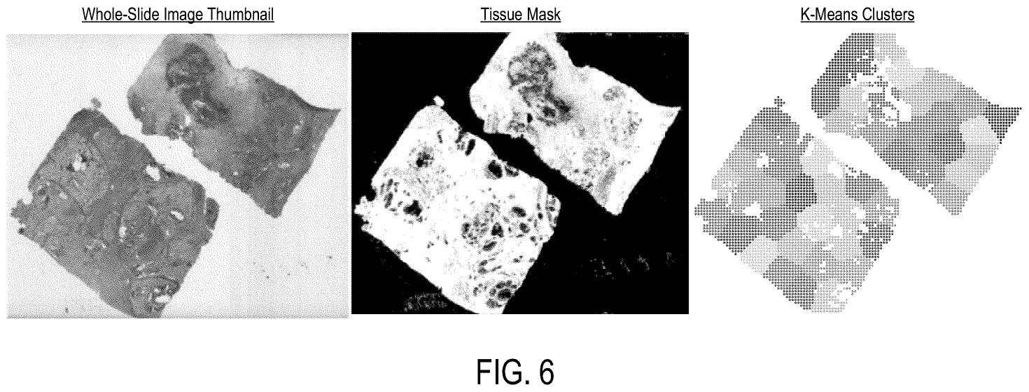

A k-means algorithm may be deployed on the location of the tiles selected previously to sample a fixed number of tiles from each WSI. The value of k was set to 49 for all experiments in an example study, though other values of k can be selected. After that, the clusters are spatially sorted based on the magnitude of the cluster centers. Spatially clustering tiles provides benefits, including that similarity is more likely to be true within a narrower radius, and clustering coordinates with two variables is computationally less expensive than high-dimensional feature vectors. The steps of the clustering algorithm are shown in FIG. 6, in which k-means clustering is applied to a whole-slide image thumbnail to create a bag of instances.

The machine learning model is then trained on the training data, as indicated at step 504. In general, the machine learning model can be trained by optimizing model parameters (e.g., weights, biases, or both) and learning other learnable variable based on minimizing or otherwise optimizing a loss function. As one non-limiting example, the loss function may be a mean squared error loss function.

Training a machine learning model may include initializing the model, such as by computing, estimating, or otherwise selecting initial model parameters (e.g., weights, biases, or both). During training, a machine learning model receives the inputs for a training example and generates an output using the initial model parameters. The model then compares the generated output with the actual output of the training example in order to evaluate the quality of the output data. For instance, the output data can be passed to a loss function to compute an error. The current machine learning model can then be updated based on the calculated error (e.g., using backpropagation methods based on the calculated error). For instance, the current model can be updated by updating the model parameters (e.g., weights, biases, or both) in order to minimize or otherwise reduce the loss according to the loss function. The training continues until a training condition is met. The training condition may correspond to, for example, a predetermined number of training examples being used, a minimum accuracy threshold being reached during training and validation, a predetermined number of validation iterations being completed, and the like. When the training condition has been met (e.g., by determining whether an error threshold or other stopping criterion has been satisfied), the current model and its associated model parameters represent the trained machine learning model. Different types of training processes can be used to adjust the model parameters and other learnable variable based on the training examples. The training processes may include, for example, gradient descent, Newton's method, conjugate gradient, quasi-Newton, Levenberg-Marquardt, among others.

In a non-limiting example, kidney WSIs, as the primary site, and their related RNA-seq data were accessed from The Cancer Genome Atlas (“TCGA”) public dataset and assembled as training data. The retrieved cases included three subtypes: clear cell carcinoma, ICD-O 8310=3, (ccRCC), chromophobe type-renal cell carcinoma, ICD-O 8317=3, (crRCC), and papillary carcinoma, ICD-O 8260=3, (pRCC). To begin, the TCGA cases were split into 80%, 10%, and 10% subsets for the training, validation, and test sets. Each case was associated with a patient and could have contained multiple diagnostic WSIs or RNA-seq files. Then, 100 bags were sampled from each WSI. As a result, the training set included 63,400 bags.

The tRNAsformer's internal representation size was set to 384. The MLP ratio and the number of self-attention heads were both four. The tRNAsformer was trained for 20 epochs with a minibatch of size 64. The AdamW was chosen as the optimizer with a starting learning rate of 3×10⁻⁴. The weight regularization coefficient was set to 0.01 to avoid overfitting. The reduce-on-plateau method was chosen for scheduling the learning rate. Therefore, the learning rate was reduced by ten every two epochs without an improvement in the validation loss. The scaling coefficient γ was set to 0.5. The last dropout layer's probability was set to 0.25. The values for the model with the lowest validation loss are reported.

The one or more trained machine learning models are then stored for later use, as indicated at step 506. Storing the model(s) may include storing model parameters (e.g., weights, biases, or both), which have been computed or otherwise estimated by training the model(s) on the training data. Storing the trained model(s) may also include storing the particular model architecture to be implemented. For instance, data pertaining to the layers in the model architecture (e.g., number of layers, type of layers, ordering of layers, connections between layers, hyperparameters for layers) may be stored.

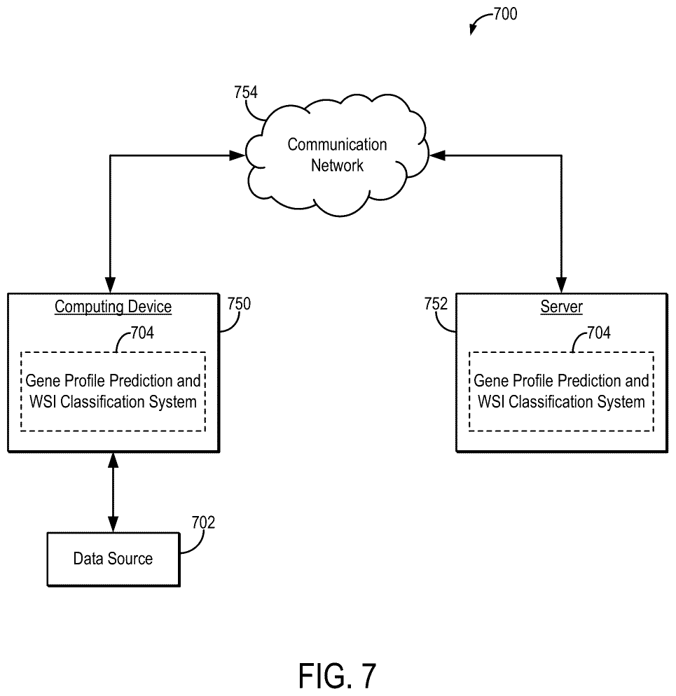

Referring now to FIG. 7, an example of a system 700 for predicting gene profile data from whole-slide images in accordance with some embodiments of the systems and methods described in the present disclosure is shown. As shown in FIG. 7, a computing device 750 can receive one or more types of data (e.g., whole-slide image data, gene expression data) from data source 702. In some embodiments, computing device 750 can execute at least a portion of a gene profile prediction and WSI classification system 704 to predict gene profile data and classify WSI data from data received from the data source 702.

Additionally or alternatively, in some embodiments, the computing device 750 can communicate information about data received from the data source 702 to a server 752 over a communication network 754, which can execute at least a portion of the gene profile prediction and WSI classification system 704. In such embodiments, the server 752 can return information to the computing device 750 (and/or any other suitable computing device) indicative of an output of the gene profile prediction and WSI classification system 704.

In some embodiments, computing device 750 and/or server 752 can be any suitable computing device or combination of devices, such as a desktop computer, a laptop computer, a smartphone, a tablet computer, a wearable computer, a server computer, a virtual machine being executed by a physical computing device, and so on. The computing device 750 and/or server 752 can also reconstruct images from the data.

In some embodiments, data source 702 can be any suitable source of data (e.g., whole-slide images, RNA-seq or other gene expression data), such as another computing device (e.g., a server storing measurement data, images reconstructed from measurement data, processed image data), and so on. In some embodiments, data source 702 can be local to computing device 750. For example, data source 702 can be incorporated with computing device 750 (e.g., computing device 750 can be configured as part of a device for measuring, recording, estimating, acquiring, or otherwise collecting or storing data). As another example, data source 702 can be connected to computing device 750 by a cable, a direct wireless link, and so on. Additionally or alternatively, in some embodiments, data source 702 can be located locally and/or remotely from computing device 750, and can communicate data to computing device 750 (and/or server 752) via a communication network (e.g., communication network 754).

In some embodiments, communication network 754 can be any suitable communication network or combination of communication networks. For example, communication network 754 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 754 can be a local area network, a wide area network, a public network (e.g., the Internet), a private or semi-private network (e.g., a corporate or university intranet), any other suitable type of network, or any suitable combination of networks. Communications links shown in FIG. 7 can each be any suitable communications link or combination of communications links, such as wired links, fiber optic links, Wi-Fi links, Bluetooth links, cellular links, and so on.

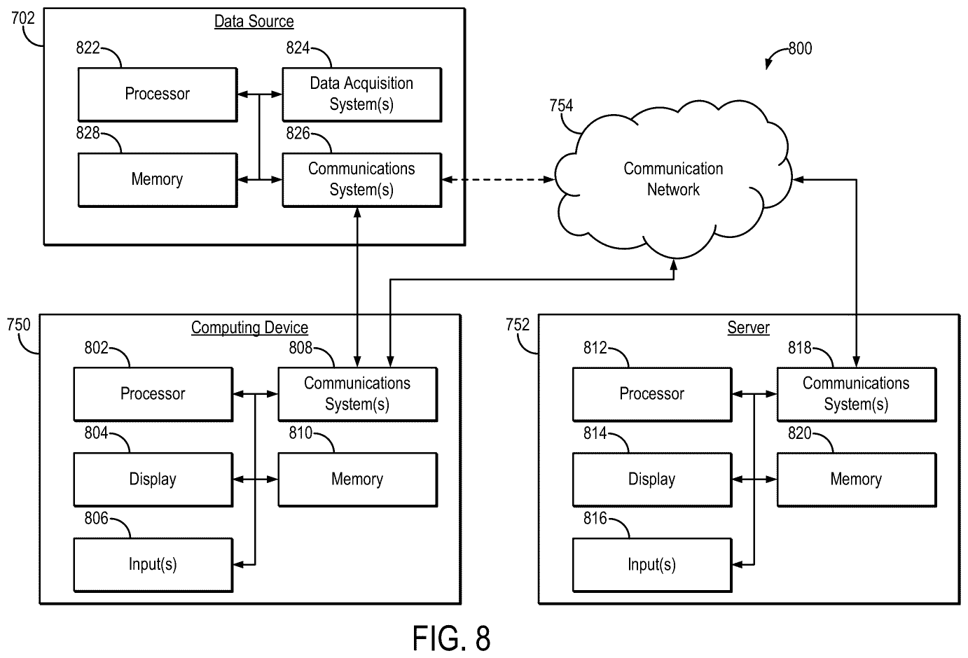

Referring now to FIG. 8, an example of hardware 800 that can be used to implement data source 702, computing device 750, and server 752 in accordance with some embodiments of the systems and methods described in the present disclosure is shown.

As shown in FIG. 8, in some embodiments, computing device 750 can include a processor 802, a display 804, one or more inputs 806, one or more communication systems 808, and/or memory 810. In some embodiments, processor 802 can be any suitable hardware processor or combination of processors, such as a central processing unit (“CPU”), a graphics processing unit (“GPU”), and so on. In some embodiments, display 804 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 806 can include any suitable input devices and/or sensors that can be used to receive user input, such as a keyboard, a mouse, a touchscreen, a microphone, and so on.

In some embodiments, communications systems 808 can include any suitable hardware, firmware, and/or software for communicating information over communication network 754 and/or any other suitable communication networks. For example, communications systems 808 can include one or more transceivers, one or more communication chips and/or chip sets, and so on. In a more particular example, communications systems 808 can include hardware, firmware, and/or software that can be used to establish a Wi-Fi connection, a Bluetooth connection, a cellular connection, an Ethernet connection, and so on.

In some embodiments, memory 810 can include any suitable storage device or devices that can be used to store instructions, values, data, or the like, that can be used, for example, by processor 802 to present content using display 804, to communicate with server 752 via communications system(s) 808, and so on. Memory 810 can include any suitable volatile memory, non-volatile memory, storage, or any suitable combination thereof. For example, memory 810 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 810 can have encoded thereon, or otherwise stored therein, a computer program for controlling operation of computing device 750. In such embodiments, processor 802 can execute at least a portion of the computer program to present content (e.g., images, user interfaces, graphics, tables), receive content from server 752, transmit information to server 752, and so on. For example, the processor 802 and the memory 810 can be configured to perform the methods described herein (e.g., the workflow illustrated in FIG. 3, the method of FIG. 4, the method of FIG. 5).

In some embodiments, server 752 can include a processor 812, a display 814, one or more inputs 816, one or more communications systems 818, and/or memory 820. In some embodiments, processor 812 can be any suitable hardware processor or combination of processors, such as a CPU, a GPU, and so on. In some embodiments, display 814 can include any suitable display devices, such as an LCD screen, LED display, OLED display, electrophoretic display, a computer monitor, a touchscreen, a television, and so on. In some embodiments, inputs 816 can include any suitable input devices and/or sensors that can be used to receive user input, such as a keyboard, a mouse, a touchscreen, a microphone, and so on.

In some embodiments, communications systems 818 can include any suitable hardware, firmware, and/or software for communicating information over communication network 754 and/or any other suitable communication networks. For example, communications systems 818 can include one or more transceivers, one or more communication chips and/or chip sets, and so on. In a more particular example, communications systems 818 can include hardware, firmware, and/or software that can be used to establish a Wi-Fi connection, a Bluetooth connection, a cellular connection, an Ethernet connection, and so on.

In some embodiments, memory 820 can include any suitable storage device or devices that can be used to store instructions, values, data, or the like, that can be used, for example, by processor 812 to present content using display 814, to communicate with one or more computing devices 750, and so on. Memory 820 can include any suitable volatile memory, non-volatile memory, storage, or any suitable combination thereof. For example, memory 820 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 820 can have encoded thereon a server program for controlling operation of server 752. In such embodiments, processor 812 can execute at least a portion of the server program to transmit information and/or content (e.g., data, images, a user interface) to one or more computing devices 750, receive information and/or content from one or more computing devices 750, receive instructions from one or more devices (e.g., a personal computer, a laptop computer, a tablet computer, a smartphone), and so on.

In some embodiments, the server 752 is configured to perform the methods described in the present disclosure. For example, the processor 812 and memory 820 can be configured to perform the methods described herein (e.g., the workflow illustrated in FIG. 3, the method of FIG. 4, the method of FIG. 5).

In some embodiments, data source 702 can include a processor 822, one or more data acquisition systems 824, one or more communications systems 826, and/or memory 828. In some embodiments, processor 822 can be any suitable hardware processor or combination of processors, such as a CPU, a GPU, and so on. In some embodiments, the one or more data acquisition systems 824 are generally configured to acquire data, images, or both, and can include a slide scanner or other digital pathology imaging system. Additionally or alternatively, in some embodiments, the one or more data acquisition systems 824 can include any suitable hardware, firmware, and/or software for coupling to and/or controlling operations of a slide scanner or other digital pathology imaging system. In some embodiments, one or more portions of the data acquisition system(s) 824 can be removable and/or replaceable.

Note that, although not shown, data source 702 can include any suitable inputs and/or outputs. For example, data source 702 can include input devices and/or sensors that can be used to receive user input, such as a keyboard, a mouse, a touchscreen, a microphone, a trackpad, a trackball, and so on. As another example, data source 702 can include any suitable display devices, such as an LCD screen, an LED display, an OLED display, an electrophoretic display, a computer monitor, a touchscreen, a television, etc., one or more speakers, and so on.

In some embodiments, communications systems 826 can include any suitable hardware, firmware, and/or software for communicating information to computing device 750 (and, in some embodiments, over communication network 754 and/or any other suitable communication networks). For example, communications systems 826 can include one or more transceivers, one or more communication chips and/or chip sets, and so on. In a more particular example, communications systems 826 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 828 can include any suitable storage device or devices that can be used to store instructions, values, data, or the like, that can be used, for example, by processor 822 to control the one or more data acquisition systems 824, and/or receive data from the one or more data acquisition systems 824; to generate images from data; present content (e.g., data, images, a user interface) using a display; communicate with one or more computing devices 750; and so on. Memory 828 can include any suitable volatile memory, non-volatile memory, storage, or any suitable combination thereof. For example, memory 828 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 828 can have encoded thereon, or otherwise stored therein, a program for controlling operation of data source 702. In such embodiments, processor 822 can execute at least a portion of the program to generate images, transmit information and/or content (e.g., data, images, a user interface) to one or more computing devices 750, receive information and/or content from one or more computing devices 750, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for predicting gene profile data from a whole-slide image using a computer system, the method comprising:
(a) accessing whole-slide image (WSI) data with the computer system, wherein the WSI data comprise whole-slide images of a histopathology sample;
(b) accessing a machine learning model with the computer system, wherein the machine learning model has been trained on training data to predict gene profile data and to classify whole-slide images;
(c) inputting the WSI data to the machine learning model using the computer system, generating as outputs gene profile data and classified WSI data, wherein the gene profile data are indicative of a predicted gene profile for the histopathology sample and the classified WSI data are indicative of a classification of the whole-slide images of the histopathology sample as one of different disease classifications; and
(d) outputting the gene profile data and classified WSI data with the computer system.

2. The method of claim 1, wherein step (a) includes:
generating WSI patch data by extracting patches from whole-slide images in the WSI data;
generating embedded WSI patch data by accessing a trained neural network and inputting the WSI patch data to the trained neural network, generating an output as the embedded WSI patch data;
forming embedded instance data from the embedded WSI patch data, wherein the embedded instance data comprises bags of instances in the embedded instance data; and
storing the embedded instance data as WSI data for inputting to the machine learning model.

3. The method of claim 2, wherein the trained neural network comprises a convolutional neural network (CNN) and generating the embedded WSI patch data comprises inputting the WSI patch data to the CNN.

4. The method of claim 3, wherein the CNN includes a DenseNet-121 architecture.

5. The method of claim 2, wherein forming the embedded instance data comprises at least one of resizing or reshaping the embedded WSI patch data into a matrix comprising blocks that each correspond to a different WSI patch embedding.

6. The method of claim 1, wherein the machine learning model comprises a transformer encoder model.

7. The method of claim 6, wherein the transformer encoder model implements an attention mechanism.

8. The method of claim 6, wherein the transformer encoder model has a first output head to output the gene profile data and a second output head to output the classified WSI data.

9. The method of claim 1, wherein the gene profile data comprise transcriptomic data.

10. The method of claim 9, wherein the transcriptomic data comprise RNA sequence (RNA-seq) data.

11. A method for generating complete genome profile data for a histopathology sample, the method comprising:
(a) accessing a whole-slide image with a computer system, wherein the whole-slide image depicts the histopathology sample;
(b) accessing an attention-based transformer encoder model with the computer system;
(c) inputting the whole-slide image to the attention-based transformer encoder model using the computer system, generating gene prediction data as an output, wherein the gene prediction data comprise a complete genome profile for the histopathology sample; and
(d) outputting the gene prediction data with the computer system.

12. The method of claim 11, wherein the attention-based transformer encoder model is a multi-head attention-based transformer encoder model comprising a first head that outputs the gene prediction data and a second head that outputs classified feature data that indicate a classification of the whole-slide image.

13. The method of claim 12, wherein the classified feature data indicate classifications of subregions of the whole-slide image.

14. A method for training a transformer encoder model to generate predicted gene profile and classified feature data from a whole-slide image, the method comprising:
(a) accessing whole-slide image data with a computer system, the whole-slide image data comprising whole-slide images that depict histopathology samples;
(b) accessing gene expression data with the computer system, the gene expression data comprising gene expressions corresponding to the histopathology samples depicted in the whole-slide images;
(c) assembling the whole-slide image data and the gene expression data into at least a training dataset using the computer system;
(d) accessing a transformer encoder model with the computer system; and
(e) training the transformer encoder model on the training dataset.

15. The method of claim 14, wherein assembling the training data set includes preprocessing the whole-slide image data to divide each whole-slide image into whole-slide image patches.

16. The method of claim 15, wherein preprocessing the whole-slide image data includes forming the whole-slide image patches into bags of instances by:
identifying tissue boundaries in each whole-slide image patch;
discarding whole-slide image patches having a percentage of pixels associated with tissue that is lower than a threshold value; and
inputting non-discarded whole-slide image patches to a clustering algorithm, generating an output as bags of instances.

17. The method of claim 16, wherein the clustering algorithm is a k-means clustering algorithm.

18. The method of claim 14, further comprising storing the trained transformer encoder model with the computer system.
